(19) United States
(12) Plant Patent Application Publication
(10) Pub. No.: US 2005/0193458 P1
(43) Pub. Date: Sep. 1, 2005

(54) GUZMANIA PLANT NAMED OF 'COMBI'

(50) GUZMANIA HYBRID; COMBI

(75) Inventors: Elly Bak, Rijsenhout (NL); Nicolaas D.M. Steur, Oude Niedorp (NL)

Correspondence Address: FOLEY AND LARDNER;SUITE 500, 3000 K STREET NW, WASHINGTON, DC, 20007, US

(73) Assignee: Corn.Bak B.V.

(21) Appl. No.: 10/788,384

(22) Filed: Mar. 1, 2004

(51) Int. Cl. A01H 5/00

(52) U.S. Cl. PLT/371

(57) ABSTRACT

A new and distinct Guzmania Plant Named ‘Combi’ particularly characterized by its solid, tenable, long lasting habit; having compact plant habit; wide leaves; superior bract production; and compound purple inflorescence.

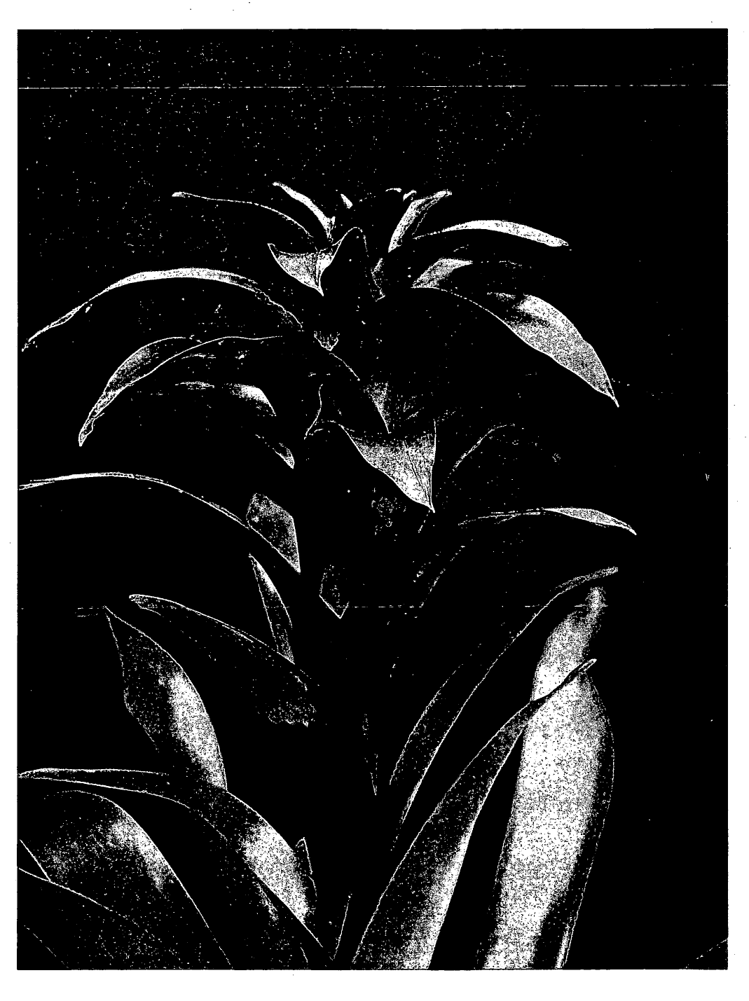

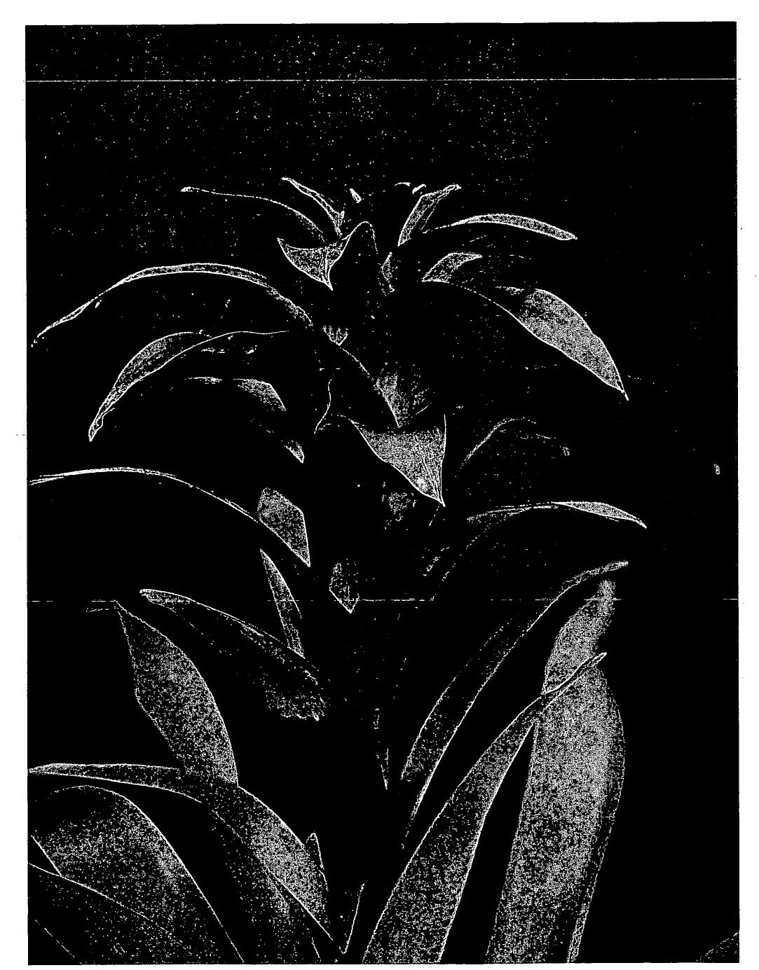

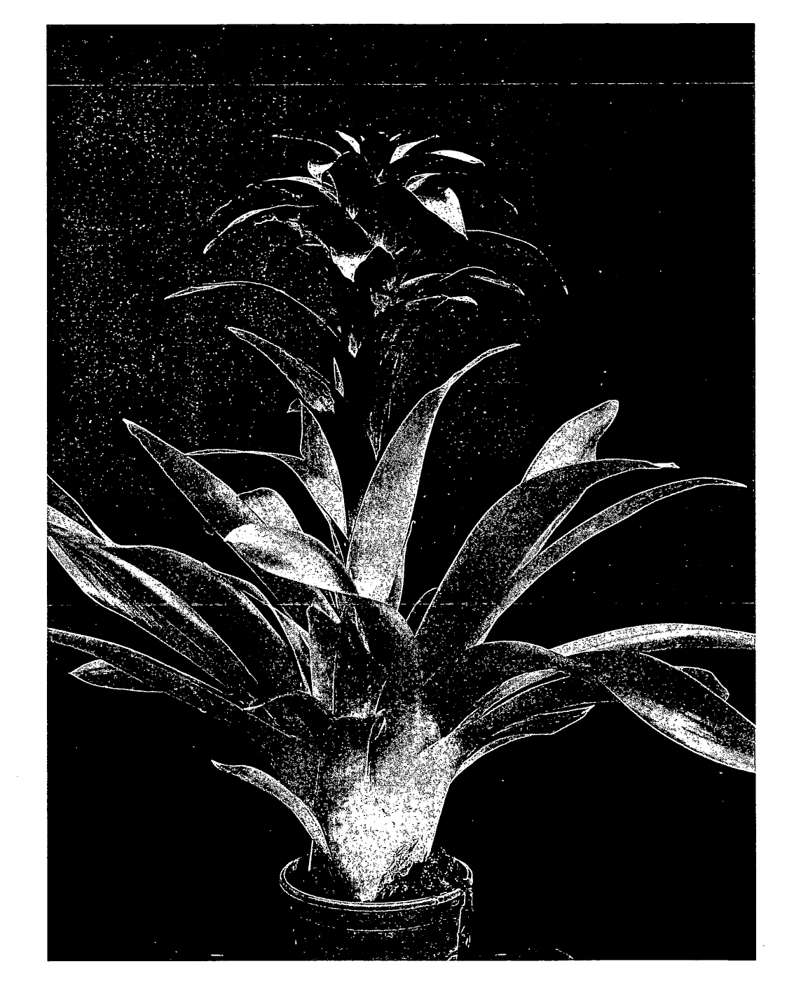

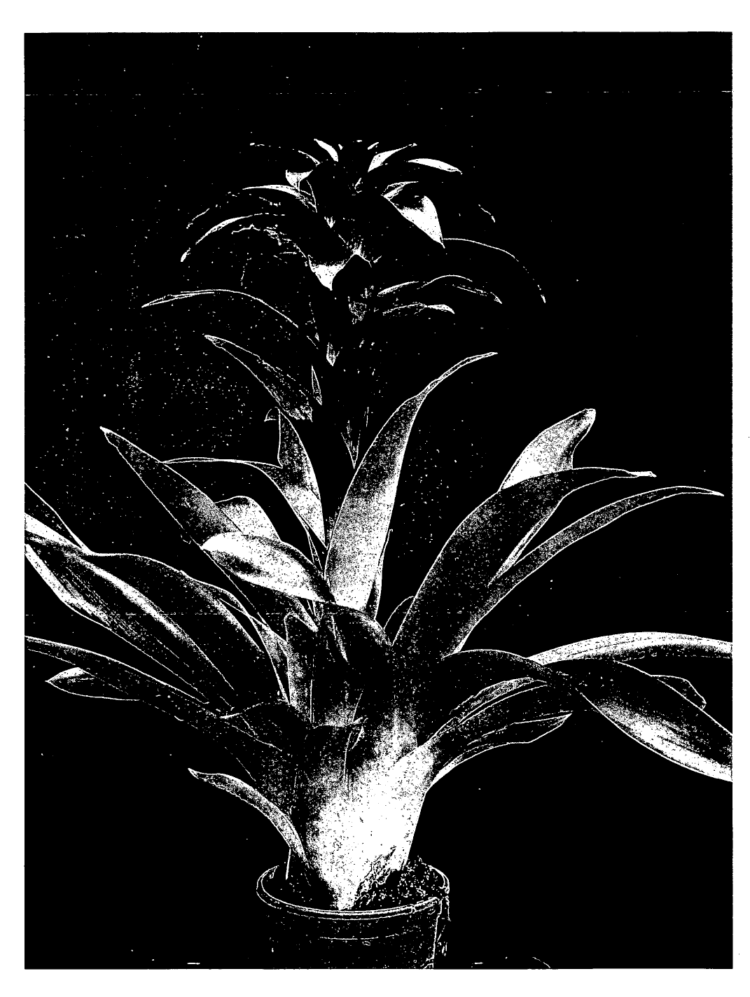

Latin name of the genus and species of the plant claimed:

Guzmania hybrid

Variety denomination:

Combi

### BACKGROUND OF THE INVENTION

The present invention relates to a new and distinct cultivar of Guzmania plant, botanically known as Guzmania hybrid, hereinafter referred to by the cultivar name ‘Combi’.

Guzmania is predominantly epiphytic with a few terrestrial species and is native to the tropics. or the most part, species vary in diameter from 7 or 8 inches to 3 or 4 feet and have rosettes of glossy, smooth-edged leaves. Floral bracts of Guzmania frequently have brilliant colors and may last for many months. The range of colors for Guzmania is generally from yellow through orange but may also include flame red and red-purple. White or yellow, tubular, three-petalled flowers may also appear on a stem or within the leaf rosette but are usually short-lived.

Guzmania may be advantageously grown as pot plants for greenhouse or home use. Desirably, the plants are shaded from direct sunlight, and during the spring to autumn period, the central vase-like part of the leaf rosette is desirably filled with water. Leaves of Guzmania are usually formed as basal rosettes, which are stiff and entire and in several vertical ranks. Guzmania plants have terminal spikes or panicles which are often bracted with petals united in a tube about as long as the calyx.

This new hybrid Guzmania ‘Combi’ is a mutation of Guzmania ‘Luna’ (unpatented) of the family Bromeliaceae, and was discovered by the inventors, Elly Bak and Nicolaas D. M. Steur. Guzmania ‘Combi’ flowered for the first time in 1999 in Assendelft, the Netherlands.

Asexual reproduction of the new cultivar through tissue culture was first performed in 1993 Assendelft, The Netherlands and has demonstrated that the combination of characteristics as herein disclosed for the new cultivar are firmly fixed and retained through successive generations of asexual reproduction.

### BRIEF DESCRIPTION OF THE INVENTION

The following traits have been repeatedly observed and are determined to be basic characteristics of ‘Combi’ which in combination distinguish this Guzmania as a new and distinct cultivar:

1. Solid, tenable, long lasting habit;

2. Compact plant habit;

3. Wide leaves;

4. Superior bract production; and

5. Compound purple inflorescence.

The closest comparison cultivar is Guzmania ‘Luna’ (unpatented). The most important differences between ‘Luna’ and ‘Combi’ are their shape and size. The inflorescence of Guzmania ‘Combi’ is more compact and the leaves are wider than those of Guzmania ‘Luna’.

Guzmania ‘Combi’ is not tested under all available environmental conditions like temperature, light intensity, day length, fertilizers and humidity.

### BRIEF DESCRIPTION OF THE DRAWING

The accompanying photographic illustrations show the new cultivar with colors as true as possible with photographic reproductions. The first photographic drawing shows a close-up of the bracts of the new cultivar ‘Combi’. The second photographic drawing shows the new cultivar ‘Combi’ in its entirety.

### DETAILED BOTANICAL DESCRIPTION

The following observations, measurements and values describe the new cultivar at two years old as grown in Assendelft, The Netherlands, under conditions which closely approximate those generally used in commercial practice. Observations, measurements and descriptions of Guzmania ‘Combi’ were done under Dutch greenhouse conditions. Color references are made to The Royal Horticultural Society Colour Chart (RHS), except where general colors of ordinary significance are used.
Plant:
   Form.—Funnel form rosette.
   Height.—Approximately 40-45 cm high (flowering).
   Growth habit.—Stemless.
   Diameter.—Approximately 60 cm.
Foliage:
   Size of leaf.—Length: approximately 45 cm; width: approximately 4-5 cm.
   Shape of leaf.—Lanceolate Surface texture: Smooth.
   Color.—Upperside: middle part RHS 147A Underside: middle part RHS 147B (Leaf color dependent on environmental conditions).
Bracts:
   Scape bracts.—Length: Lowest bract approximately 20 cm, to approximately 12 cm just below the primary bracts Width: Approximately 4-4.5 cm General shape: Lanceolate Number: Approximately 12 Texture: Smooth Color: RHS 187D (both surfaces) with green tips, RHS 139A.
   Primary bracts.—Length: Lowest bract approximately 12 cm, to approximately 8 cm at the top Width: Approximately 4-5 cm General shape: Lanceolate Number: Approximately 8 Texture: Smooth Color: RHS 187D (both surfaces).
   Floral bracts.—Length: Approximately 2-5 cm Width: Approximately 1 cm Number: About 7 per branch
Flowers:
   Borne.—Erect.
   Shape of inflorescence.—Compound.
   Size of inflorescence on stalk.—Approximately 9 cm.
   Diameter of inflorescence.—Approximately 20 cm.
   Individual petals.—(disposed within the inflorescence) Length: Approximately 5 cm Width: Approximately 0.5 cm Quantity: Approximately 45 flowers depending on the size of the plant.
   Color.—Unknown since the flowers are disposed within the inflorescence.
Time of blooming: A fully grown plant can produce an inflorescence containing approximately 45 flowers and can bloom anytime throughout the year starting approximately 15 weeks after natural induction or induction with acetylene. The total length of blooming of the whole inflorescence is 5 weeks.
Reproductive organs:
   Ovaries.—Superior.
   Stamens.—6 in number.
   Seed characteristics.—The plant is a sterile hybrid, therefore no fruit or seed.
Disease/pest resistance/susceptibility.—No observations made to date.

1. A new and distinct Guzmania plant named ‘Combi’, as described and illustrated herein.
